(19) United States
(12) Patent Application Publication
(10) Pub. No.: US 2004/0267179 A1
(43) Pub. Date: Dec. 30, 2004

(54) Knee unloading orthotic device and method

(76) Inventor: Max Lerman, Beverly Hills, CA (US)

Correspondence Address: Ryndak & Suri, Suite 2630, 30 North LaSalle Street, Chicago, IL, 60602, US

(21) Appl. No.: 10/609,986

(22) Filed: Jun. 30, 2003

(51) Int. Cl. A61F 5/00

(52) U.S. Cl. 602/26; 602/16

(57) ABSTRACT

A knee brace is provided having an upper leg support, a lower leg support, a hinge mechanism connecting the leg supports and knee straps which cross the knee and apply a three-point corrective unloading force to a degenerated compartment of the knee joint. The lower leg support includes upper and lower arms pivotally attached to each other allowing angular adjustment of the knee brace in the coronal plane to provide a varus/valgus correction to the wearer's leg. The hinge mechanism may be aligned with the wearer's forward progression by rotationally adjusting the upper and lower leg supports relative to the hinge mechanism.

### FIELD OF THE INVENTION

The present invention relates to a knee brace having knee straps that apply a corrective force to the knee joint through a three-point pressure pattern. The lower leg support of the knee brace includes upper and lower arms pivotally attached to each other allowing angular adjustment of the knee brace in the coronal plane to provide varus/valgus correction to the wearer's leg.

### BACKGROUND OF THE INVENTION

The human knee is prone to a number of diseases that can affect a person's ability to walk without pain. Osteoarthritis, also known as degenerative knee joint disease, is the breakdown of cartilage in the knee joint. Degeneration of cartilage causes unbalanced loading on the medial or lateral knee compartment between the condyles of the femur and the tibial plateau. With advancement of the disease, the space between the femur and tibia decreases. Left unattended, the disease may advance to such an extent that the space is eliminated and abrasion between the femur and tibia occurs. Not only does this cause pain but the disease may also propagate a varus or valgus deformity sufficient to hamper or prevent ordinary ambulation.

Known are orthotic knee devices that mechanically stabilize and unload the knee. Conventional knee unloading orthotic devices, however, are subject to several drawbacks. Many present knee unloading orthotic devices require custom manufacture in order to provide the proper corrective force to the patient's knee. Other orthotic knee devices are limited in their unloading capability. Present braces utilizing a condyle pad or hinge device in intimate contact with the knee joint tend to restrict knee separation movement, for example. Moreover, conventional orthotic devices fail to provide an adequate remedy for the simultaneous treatment of unicompartmental knee degeneration and misalignment between the femur and tibia.

A need therefore exists for an orthotic device that does not restrict the separation between the femur and tibia when imparting a corrective unloading force to a degenerated knee compartment. A need further exists for an orthotic device capable of applying unicompartmental unloading as well as varus/valgus correction to a patient's leg.

### SUMMARY OF THE INVENTION

In accordance with the present invention, a knee unloading orthotic device is provided which utilizes a three-point pressure pattern to unload a degenerated knee compartment to relieve pain, stabilize the knee and improve ambulation. The device includes an upper leg support which is secured to the upper leg of a person and lower leg support which is secured to the lower leg, a hinge mechanism connecting the upper and lower leg supports, and first and second knee straps. The knee unloading orthotic device is applied to a person's leg so that the hinge mechanism is proximate the knee compartment to be unloaded. One end of the first knee strap attaches to the upper leg support. The first knee strap extends above the knee and wraps around the back of the knee and a second end of the first knee strap attaches to the lower leg support. In a similar manner, one end of the second knee strap is attached to the lower leg support. The second knee strap extends under the kneecap and around the back of the knee and a second end of the second knee strap attaches to the upper leg support. The first and second knee straps intersect proximate the opposing knee compartment to provide a corrective unloading force to the knee compartment proximate the hinge mechanism. The knee unloading orthotic device produces a three-point pressure pattern between the upper leg support, the lower leg support and the point where the first and second knee straps intersect. The tension provided by stretching the knee straps across the knee urges or pulls the femur and tibia toward the pressure point where the knee straps cross. The knee is urged or pulled toward the hinge mechanism by the upper and lower leg support pressure points. A clearance space or gap is maintained between the knee unloading device and the knee permitting full separation in the degenerated knee compartment.

The knee unloading orthotic device of the present invention may be applied medially or laterally to a person's leg. The knee straps are preferably made of a stretchable or expandable material. The hinge mechanism defines a hinge side of the device and the second ends of the first and second knee straps are preferably attached to the hinge side of the lower and upper leg supports, respectively. This increases the tension across the knee straps increasing the corrective unloading force imparted onto the knee.

In a preferred embodiment of the present invention, the upper leg support includes an upper elongated member and an upper leg cuff and the lower leg support includes a lower elongated member and a lower leg cuff. The upper leg cuff is pivotally attached to the upper elongated member and the lower leg cuff is pivotally attached to the lower elongated member, each cuff being securable in a desired fixed position relative to its respective upper or lower elongated member. This enables the knee unloading orthotic device to be adjusted so that the hinge mechanism may be aligned with the forward progression of the wearer's center of gravity. Preferably, the upper leg support includes a thigh strap secured to the upper elongated member. The thigh strap may then be wrapped around the upper leg of the wearer to secure the upper leg cuff to the upper leg. In a similar manner, the lower leg support preferably includes a calf strap attached to the lower elongated member. The calf strap may be wrapped around the wearer's lower leg to secure the lower leg cuff to the lower leg.

In an alternate embodiment of the present invention, the upper and lower leg cuffs include an inner skin adhesion layer. The skin adhesion layer promotes frictional adhesion between the skin on the wearer's leg and the leg cuffs. Preferably, the skin adhesion layer is made from a silicon-based material that can be worn against the skin for extended periods of time without irritation or discomfort.

The upper and lower elongated members may each further include a slot extending along a portion of the length of each elongated member. The upper slot permits the upper leg cuff to be adjustably positioned along a portion of the length of the upper elongated member. Likewise, the lower slot permits the lower leg cuff to be adjustably positioned along a portion of the length of the lower elongated member. The knee unloading orthotic device of the present invention may thereby be adapted to properly fit a wide array of patients from children to adult patients regardless of gender. The hinge mechanism may include a plurality of openings into which one or two stop pins may be inserted to restrict flexion or extension movement as desired.

In accordance with another aspect of the present invention, a single knee strap is wrapped around the knee and attached to the hinge sides of the upper and lower leg supports to provide a three-point pressure pattern. The lower leg support includes an upper arm pivotally attached to a lower arm by a pivot joint permitting selective adjustment of the lower arm relative to the upper arm. When the knee unloading orthotic device is applied to the leg, the angular adjustment of the upper and lower arms occurs in the coronal plane providing a varus/valgus correction as well as an unloading force to the leg of the person. Alternatively, the second knee strap attached to the lower leg support may be extended to wrap around the knee and be affixed to the upper leg support in order to provide a greater corrective force. In addition, the upper leg cuff may be pivotally adjusted relative to the upper elongated member and the lower leg cuff may be pivotally adjusted relative to the lower elongated member allowing the hinge mechanism to be aligned with the person's forward progression as previously described.

Preferably, the pivot joint has associated with it structure for lateral angular adjustment of one of the upper and lower arms relative to the other arm and the pivot joint, which in one embodiment includes intermeshing teeth for incremental angular adjustment of the upper and lower arms. A suitable structure is provided for securing the arms in a fixed position, which structure may be configured so that the pivot joint includes a screw which engages the opposing intermeshing teeth to securely lock the arms in a fixed relative lateral angular position when the screw is tightened. Loosening the screw allows the arms to be adjusted. The adjustment structure may omit intermeshing teeth allowing continuous or infinite lateral angular adjustment of the lower arm relative to the upper arm.

In accordance with another aspect of the invention, a method is provided for applying a corrective unloading force with a knee brace to a knee compartment of a person. The knee brace includes an upper leg support, a lower leg support and a hinge mechanism pivotally connecting the upper and lower leg supports along a hinge side to permit flexion and extension movement of the person's knee. The hinge is positionable adjacent a first knee compartment. The method includes positioning the brace so that the hinge is proximate the first knee compartment, extending a first knee strap preferably from the hinge side of the upper leg support along the top of the knee and around the back of the knee and preferably to the hinge side of the lower leg support, and extending a second knee strap preferably from the hinge side of the lower leg support along the bottom of the knee and around the back of the knee and preferably to the hinge side of the upper leg support, the second knee strap intersecting the first knee strap proximate a second knee compartment.

In accordance with another aspect of the present invention, a method is provided for applying a corrective unloading force to a knee compartment and a varus/valgus correction to the leg of a person by applying a knee brace to the person's leg. The knee brace includes an upper leg support, a lower leg support having an upper arm, a lower arm and a pivot joint connecting the upper and lower arm, and a hinge mechanism connecting the upper leg support and the lower leg support along a hinge side of the brace. The hinge mechanism permits flexion and extension knee movement and is positionable proximate a first knee compartment. The method includes positioning the brace so that the hinge is proximate the first knee compartment, adjusting the lower arm relative the upper arm to provide a desired angular position between the upper and lower arms in the coronal plane to impart a varus/valgus correction, and extending a first knee strap preferably from the hinge side of the upper leg support proximate the front of the knee and around the back of the knee to the hinge side of the lower leg support.

In accordance with another aspect of the present invention, a method is provided for applying a corrective unloading force to a knee compartment by applying a knee brace to the leg of the person. The knee brace includes an upper elongated member pivotally attached to an upper leg cuff, a lower elongated member pivotally attached to a lower leg cuff, and a hinge mechanism attaching the upper elongated member to the lower elongated member along a hinge side to permit flexion and extension movement of the person's knee. The hinge mechanism is positionable proximate a first knee compartment. The method includes positioning the brace so that the hinge is proximate the first knee compartment, aligning the hinge mechanism with the person's forward progression by rotationally positioning the upper leg cuff relative the upper elongated member and the lower leg cuff relative the lower elongated member, and extending a first knee strap preferably from the hinge side of the upper leg support proximate the front of the knee and around the back of the knee preferably to the hinge side of the lower leg support.

In accordance with another aspect of the invention, a method is provided for encouraging the proper alignment of a leg by applying a knee brace to a person's leg. The knee brace comprises an upper elongated member pivotally attached to an upper leg cuff, a lower elongated member pivotally attached to a lower leg cuff and a hinge mechanism attaching the upper elongated member to the lower elongated member along a hinge side to permit flexion and extension movement of the person's knee. The hinge mechanism is positionable proximate a first knee compartment and the lower elongated member has an upper arm pivotally attached to a lower arm. The method comprises positioning the brace so that the hinge is proximate the first knee compartment, adjusting the lower arm relative the upper arm to provide a desired angular position between the upper and lower arms to provide a varus/valgus correction to the person's leg, aligning the hinge mechanism with the person's forward progression by rotationally positioning the upper leg cuff relative the upper elongated member and the lower leg cuff relative the lower elongated member and extending a first knee strap preferably from the hinge side of the upper leg support proximate the front of the knee and around the back of the knee preferably to the hinge side of the lower leg support.

### BRIEF DESCRIPTION OF THE DRAWINGS

FIG. 1 is a fragmentary front perspective view of the knee unloading orthotic device applied to a person's leg in accordance with the present invention;

FIG. 2 is a fragmentary exploded perspective view of the knee unloading orthotic device of FIG. 1;

FIG. 3 is a fragmentary view taken along line 3-3 of FIG. 1;

FIG. 4A is a schematic view of a varus deformity;

FIG. 4B is a schematic view of the correction imparted by the knee unloading orthotic device of FIG. 1 to a varus deformity;

FIG. 4C is a fragmentary front perspective view of the knee unloading orthotic device of FIG. 1 applied medially to a person's leg in accordance with the present invention;

FIG. 5A is a schematic view of a valgus deformity;

FIG. 5B is a schematic view of the correction imparted by the knee unloading device of FIG. 1 to a valgus deformity;

FIG. 5C is a fragmentary front perspective view of the knee unloading device of FIG. 1 applied laterally to a person's leg in accordance with the present invention; and

FIG. 6 is a side perspective view of the knee unloading orthotic device of FIG. 1.

### DETAILED DESCRIPTION OF THE PREFERRED EMBODIMENTS

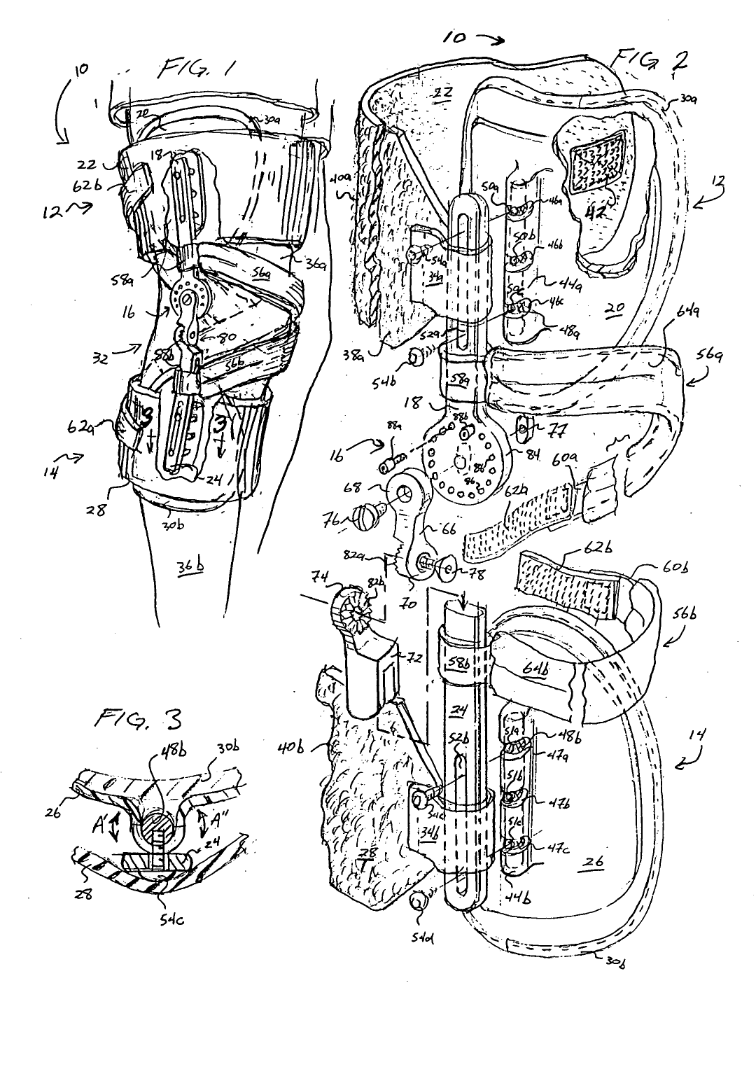

Referring to the FIGURES generally, where like reference numerals denote like structure and elements, and in particular to FIGS. 1 and 2, a knee unloading orthotic device 10 is shown and includes an upper leg support 12, a lower leg support 14 and a hinge mechanism 16 pivotally connecting the upper leg support 12 to the lower leg support 14. Upper leg support 12 includes an upper elongated member 18, attached to an upper leg cuff 20 and a thigh strap 22. Lower leg support 14 includes a lower elongated member 24 attached to a lower leg cuff 26 and a calf strap 28. Upper and lower leg cuffs 20 and 26 each have a respective inner liner 30a and 30b. Upper and lower leg cuffs 20 and 26 are preferably made of a lightweight bendable polymer or plastic material. The contoured shape of upper and lower cuffs 20 and 26 provide a comfortable and accommodating fit when liners 30a and 30b are placed in contact with a person's upper and lower leg, respectively.

One end of thigh strap 22 and one end calf strap 28 are respectively attached to upper leg cuff 20 and lower leg cuff 26 with fasteners 34a and 34b. Fasteners 34a and 34b are preferably made from a flexible textile material and form a closed loop, the loop having sufficient inner circumference to wrap around and secure to upper and lower elongated members 18 and 24, respectively. Thigh strap 22 is preferably made of a flexible and expandable material and is suitably adapted to wrap around the upper leg or thigh 36a of a person. Thigh strap 22 has an outer surface 38a preferably lined in suitable locations with hook or loop fastening material 40a, such as Velcro®. Affixed to the opposing end of thigh strap 22 on the interior surface thereof in suitable locations is a pad or area 42 of complementary loop or hook fastening material for securing to outer surface 38a. Thigh strap 22 is of sufficient length to enable pad 42 to overlap and attach to a portion of thigh strap 22 when wrapped around a person's thigh 36a. Pad 42 attaches to hook or loop material 40a to secure upper leg support 12 to thigh 36a. Alternatively, any suitable attachment structure could be utilized for thigh strap 22, such as a buckle arrangement.

Calf strap 28 is essentially identical to thigh strap 22 and wraps around lower leg or calf 36b to secure lower leg support 14 to calf 36b. Preferably, thigh and calf straps 22 and 28 are made of an expandable or stretchable material to provide a firm friction fit with the wearer's leg with neoprene being preferred. The inner surface of thigh and calf straps 22 and 28 may be textured to further promote frictional adherence to the person's leg. It has been found that contacting liners 30a and 30b directly to the skin of upper and lower legs, respectively, provides the best adherence of device 10 on the wearer's leg.

Upper elongated member 18 and lower elongated member 24 are pivotally attached to each other by hinge mechanism 16. Hinge mechanism 16 thereby allows flexion and extension movement of the knee when device 10 is applied to a person's leg. Hinge mechanism 16 and upper and lower elongated members 18 and 24 define a hinge side of device 10. Device 10 is placed on a person's leg so that hinge mechanism 16 is proximate the degenerative knee compartment.

In one embodiment of the present invention, upper and lower elongated members 18 and 24 are rotatably mounted relative to respective upper and lower leg cuffs 20 and 26 and can be fixed in a desired position relative to cuffs 20 and 26. Any suitable structure can be utilized for such mounting and fixing in a desired position. A suitable structure for mounting can be molded into the outer central portion of upper leg cuff 20 as an outwardly protruding envelope or pocket 44a. Envelope 44a is generally cylindrical in shape and has at least one and preferably a plurality of spaced apart arcuate slots 46a, 46b and 46c which extend laterally across a portion of the outer circumference of envelope 44a exposing the interior of envelope 44a. A receiving member 48a is disposed within envelope 44a and includes seatings 50a, 50b and 50c corresponding to and exposed by slots 46a, 46b and 46c. Receiving member 48a is elongated and cylindrical in shape so as to fit within envelope 44a in a complementary manner and is freely movable or otherwise rotatable within envelope 44a. Alternatively, for example, a ball and socket arrangement could be utilized with a suitable mechanism for locking the leg cuff in a desired orientation relative to the upper or lower elongated member.

Upper elongated member 18 has a narrow elongated opening 52a that extends along a portion of the length of elongated member 18. Upper elongated member 18 is aligned with envelope 44a so that screws 54a and 54b may be inserted through opening 52a to be received by any combination of two seats 50a, 50b or 50c. This attachment configuration enables upper elongated member 18 to be rotatably positioned anywhere along the arcuate extent of slots 46a-46c. Narrow elongated opening 52a also enables upper leg cuff 20 to be selectively positioned along the axis of upper elongated member 18 to ensure proper fit of device 10 on a person's leg. This is advantageous as device 10 may thereby be adapted to accommodate legs of varying lengths.

Similarly, an outwardly protruding envelope 44b is molded into the outer central portion of lower leg cuff 26. Envelope 44b has a plurality of spaced apart arcuate slots 47a, 47b, and 47c which extend laterally across a portion of the outer circumference of envelope 44b exposing the interior of envelope 44b. A receiving member 48b is disposed within envelope 44b and includes seatings 51a, 51b and 51c corresponding to and exposed by slots 47a, 47b and 47c. Receiving member 48b is elongated and cylindrical in shape so as to fit within envelope 44b in a complementary manner and is freely movable within envelope 44b.

Lower elongated member 24 has a narrow elongated opening 52b that extends along a portion of the length of lower elongated member 24. Lower elongated member 24 is aligned with envelope 44b so that screws 54c and 54d may be inserted through opening 52b to be received by any combination of two seats 51a, 51b or 51c. This attachment configuration enables lower elongated member 24 to be rotatably positioned anywhere along the arcuate extent of slots 47a-47c. Arrows A′ and A″ in FIG. 3 illustrate the rotational range of motion between lower elongated member 24 and lower leg cuff 26. It is understood that upper elongated member 18 has a similar rotational range of motion relative to upper leg cuff 20. Elongated opening 52b enables lower leg cuff 26 to be adjustably positioned along the axis of lower elongated member 24.

Device 10 is applied to the person's leg such that hinge mechanism 16 is positioned proximate the degenerative knee compartment. The capability to rotate the leg cuffs relative to each respective elongated member enables device 10 to be adjusted in order to maintain hinge mechanism 16 in alignment with the forward progression of the body's center of gravity during the gait cycle. This is advantageous as proper alignment of hinge mechanism 16 with the body's forward progression improves the unloading capability of device 10 and promotes comfortable wear of device 10.

In one embodiment of the present invention, knee straps provide a corrective unloading force to the degenerative knee compartment. One end of a first knee strap 56a is secured to upper leg support 12 by any suitable fastening structure which can be, for example, by a buckle or with hook and loop fastening material such as Velcro®. Preferably, a loop material fastener 58a is attached to an end of first knee strap 56a and is wrapped around upper elongated member 18. Loop material fastener 58a may alternatively comprise hook material. First knee strap 56a is made of a flexible, resilient expandable material. Knee strap 56a is then stretched or pulled to extend above the wearer's knee and is wrapped around the opposing side of the knee and around the back of the knee where a second end 60a of knee strap 56a attaches to lower leg support 14. Second end 60a preferably includes a strip of hook or loop fastening material 62a which attaches to complementary hook or loop material 40b lining at least a portion of the exterior of calf strap 28 on the hinge side of device 10.

In a similar fashion, a second knee strap 56b, which is essentially identical to first knee strap 56a, is secured to lower leg support 14 using hook and loop material such as Velcro® or any other suitable fastening structure, such as a buckle arrangement. Preferably, a loop material fastener 58b attached to an end of second knee strap 56b is wrapped around lower elongated member 24. Loop material fastener 58b may alternatively comprise a hook material. Knee strap 56b is then stretched or pulled to extend below the wearer's knee and wraps around the opposing side of the wearer's knee around the back of the knee where a second end 60b of knee strap 56b attaches to upper leg support 12. Second end 60b preferably includes a strip of hook or loop fastening material 62b which attaches to complementary hook or loop material 40a lining at least a portion of the exterior of thigh strap 22 on the hinge side of device 10. First and second knee straps 56a and 56b preferably include a respective padded sleeve 64a and 64b which envelopes a substantial portion of each respective knee strap providing additional comfort to the wearer of device 10.

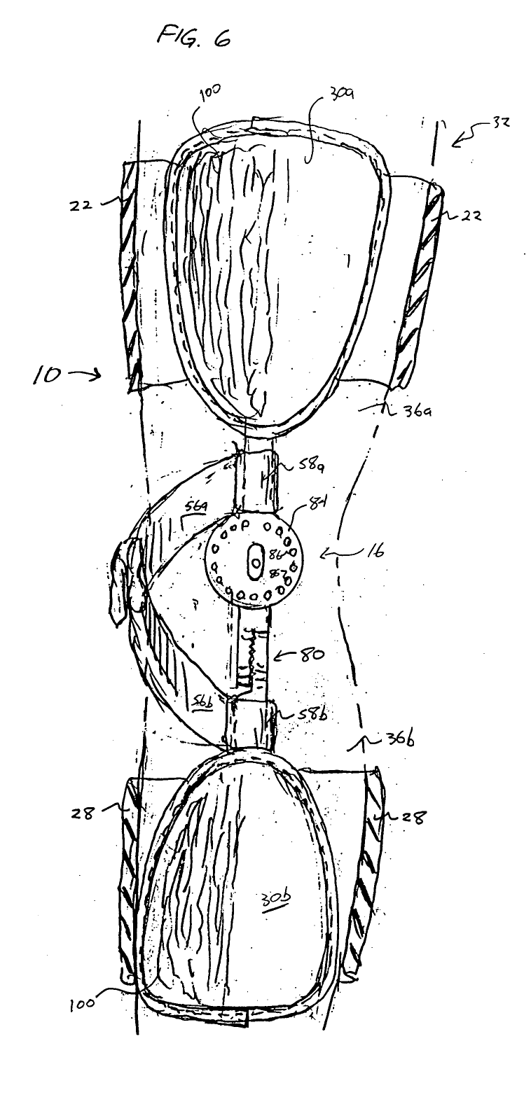

First and second knee straps 56a and 56b cross or otherwise intersect proximate the opposing knee compartment (i.e., if the medial compartment is degenerated, then the knee straps cross proximate the lateral knee compartment and vice versa) to create a three-point pressure pattern which separates the femoral condyle from the tibial plateau substantially reducing or eliminating the frictional pressure in the degenerated knee compartment. For example, lateral application of device 10 provides a corrective unloading force to the medial knee compartment as shown in FIGS. 5A-5C. The crossing of first and second knee straps 56a and 56b provides a pressure point which pulls the femur and tibia medially while upper and lower leg cuffs 20 and 26 provide pressure points which pull the knee laterally toward hinge mechanism 16. The gap between the knee and hinge mechanism 16 provides ample clearance space for the knee, femur and tibia to move in response to these forces. Full medial separation occurs uninhibited by any component of device 10. Device 10 thereby reduces the pain associated with osteoarthritis and increases mobility for the wearer of device 10. Device 10 may be worn either medially or laterally to impart a respective lateral or medial compartmental separation. The crossing aspect of the two knee straps carries the benefit of added comfort to the wearer of device 10 and eliminates the need for a condyle pad or similar component.

In another embodiment of the present invention, device 10 may be used to correct a varus/valgus deformity. In this embodiment, lower leg support 14 further includes an upper arm 66 having a pivot head 68 and a first pivot plate 70, and a lower arm 72 having a second pivot plate 74 as shown in FIG. 2. It is understood that lower arm 72 and lower elongated member 24 may be the same component. A screw 76 and a nut 77 secure pivot head 68 to hinge mechanism 16 providing pivotal rotation between hinge mechanism 16 and upper arm 66. First pivot plate 70 is offset 900 from pivot head 68. A screw 78 connects first pivot plate 70 to second pivot plate 74 to form a pivot joint 80. First pivot plate 70 has a plurality of teeth 82a which intermesh with a plurality of teeth 82b of second pivot plate 74 to selectively lock pivot joint 80 when screw 78 is tightened. Pivot joint 80 thereby provides incremental adjustment of lower arm 72 relative to upper arm 66. One of ordinary skill in the art will recognize that first and second pivot plates may lack intermeshing teeth thereby providing continuous lockable adjustment between upper arm 66 and lower arm 72. When device 10 is applied to the leg of a person, pivot joint 80 provides angular adjustment between upper and lower arms 66 and 72 in the coronal plane or the plane that divides the body into anterior and posterior portions.

The lower end of upper elongated member 18 preferably includes an enlarged circular portion 84 having a plurality of threaded openings 86 spaced apart along the circumference thereof. Threaded stop pins 88a and 88b may be inserted into selected openings 86 to restrict flexion and/or extension movement of the wearer's knee.

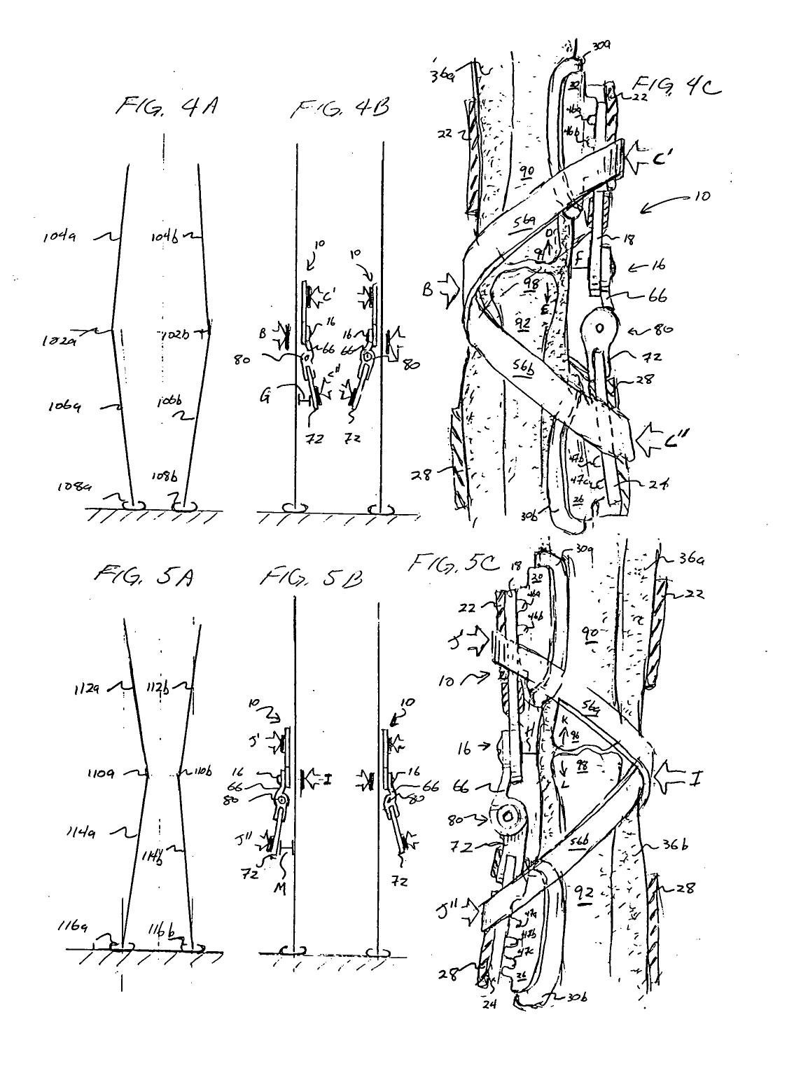

In many instances osteoarthritis is a result of or further propagates an angular deformity between the femur and tibia. Such varus/valgus deformities may cause a deviation in the amount of contact pressure produced between the femur and the tibia, leading to further degeneration of the knee joint. In a varus deformity, otherwise known as bow-legged deformity, the legs are laterally directed in alignment as shown in FIG. 4A. As a result of the degenerative disease, knee 102a is unable to maintain proper alignment between upper leg 104a and lower leg 106a when weight or pressure is applied to foot 108a. Similarly, knee 102b is also unable to maintain proper alignment between upper leg 104b and lower leg 106b when weight or pressure is applied to foot 108b. Knees 102a and 102b are consequently pushed laterally outward, providing a bowlegged appearance. To correct this deformity, device 10 is applied medially as shown in FIG. 4B. Lower arm 72 is adjusted to extend medially as shown in FIG. 4B. Knee straps 56a and 56b are extended about and around the knee as previously described. In this configuration, device 10 imparts a corrective unloading force to the medial knee compartment as well as a varus correction as shown in FIGS. 4B and 4C.

FIGS. 4B and 4C illustrate the three-point pressure pattern imparted by device 10. The clearance between the knee and device 10 depicted by length F allows the tension imparted by crossing knee straps 56a and 56b at point B to pull femur 90 and tibia 92 to the left. The tension force at pressure points C′ and C″ pull the knee joint to the right toward hinge mechanism 16. These opposing forces impart a medial separation by pulling femoral medial condyle 94 upward as indicted by arrow D while simultaneously pulling tibial plane 98 downward as indicated by arrow E.

Concomitantly, device 10 corrects the varus deformity as shown schematically in FIG. 4B. Pivot joint 80 is adjusted to medially extend lower arm 72 to a desired corrective position relative to upper arm 66. This creates a clearance space or gap between device 10 and tibia 92 as denoted by length G. Just as clearance F between the knee and hinge mechanism 16 enables the knee to be pulled toward the hinge mechanism, clearance G enables the tibia to be pulled to the right by pull force C″, thereby bringing the femur and tibia into normal alignment as shown in FIG. 4B. FIG. 4B further illustrates that device 10 may be applied to either leg to correct a varus deformity.

In a valgus deformity, otherwise known as knocked-knee deformity, the legs are medially directed in alignment as shown in FIG. 5A. As a result of the degenerative disease, knee 110a is unable to maintain proper alignment between upper leg 112a and lower leg 114a when weight or pressure is applied to foot 116a. Similarly, knee 110b is unable to maintain proper alignment between upper leg 112b and lower leg 114b when weight or pressure is applied to foot 116b. Knees 110a and 110b are consequently pushed medially inward, providing a knocked-knee appearance. To correct this deformity, device 10 is applied laterally as shown in FIG. 5B. Lower arm 72 is adjusted to extend laterally as shown in FIG. 5B. Knee straps 56a and 56b are extended about and around the knee as previously described. In this configuration, device 10 imparts a corrective unloading force to the lateral knee compartment as well as a valgus correction as shown in FIGS. 5B and 5C.

FIGS. 5B and 5C illustrate the three-point pressure pattern imparted by lateral application of device 10. The clearance between the knee and device 10 depicted by length H allows the tension between crossing knee straps 56a and 56b and upper and lower leg supports 12 and 14 to unload and align the person's leg. The pressure at point I pulls femur 90 and tibia 92 to the right. The pressure at points J′ and J″ pulls the knee to the left toward hinge mechanism 16. These opposing forces impart a lateral separation by pulling femoral lateral condyle 96 upward as indicted by arrow K while simultaneously pulling tibial plane 98 downward as indicated by arrow L.

The valgus deformity is corrected as shown schematically in FIG. 5B. Pivot joint 80 is adjusted to laterally extend lower arm 72 to a desired corrective position relative to upper arm 66. This creates a clearance space or gap between device 10 and the tibia denoted by length M. Gap M enables the tibia to be pulled to the left, thereby bringing the femur and tibia into normal alignment as shown in FIGS. 5B and 5C. FIG. 5B further illustrates that device 10 may be applied to either leg to correct a valgus deformity.

The amount of corrective force imparted by device 10 on a wearer's leg may be adjusted in a variety of ways. The degree to which each knee strap is extended will affect the amount of corrective force applied to the leg. Also, the attachment site of second ends 60a and 60b to respective lower and upper leg supports 14 and 12 further affects the corrective force applied to the leg. Preferred is attachment of strip 62a onto the hinge side of calf strap 28 and attachment of strip 62b to the hinge side of thigh strap 22. This maximizes the tension across the knee straps. Also, adjusting the angle between upper and lower arms 66 and 72 to increase the size of gaps G and M will increase the tension across the knee straps thereby increasing the corrective force imparted by device 10.

The provision of two knee straps produces a greater corrective force as opposed to prior art knee unloading devices, which employ a single strap. This is particularly advantageous in severe compartmental degradation situations wherein a high degree of tension across the knee straps is required to impart an adequate varus/valgus correction and concomitantly unload the knee. On the other hand, situations may exist wherein a lesser degree of correction is necessary. For example, device 10 may be used to correct a mild case of unicompartmental osteoarthritis and/or a small varus/valgus correction by utilizing a single knee strap. Alignment of the hinge mechanism with the wearer's forward progression may be accomplished regardless the magnitude and type of correction imparted by device 10.

FIG. 6 illustrates a preferred embodiment of the present invention wherein surfaces of inner liners 30a and 30b are composed of a layer 100 having a slightly adhesive character to promote frictional adhesion between liners 30a and 30b and the skin of the person wearing device 10. Preferably, layer 100 is a substantially inert silicone-based material that can be worn against the skin for extended periods of time without irritation or discomfort.

While the invention has been described with respect to certain preferred embodiments, as will be appreciated by those skilled in the art, it is to be understood that the invention is capable of numerous changes, modifications and rearrangements and such changes, modifications and rearrangements are intended to be covered by the following claims.

1. A knee brace to be worn by a person for unloading pressure to the person's knee having first and second knee compartments comprising:
an upper leg support and a lower leg support;
a hinge connecting the upper and lower leg supports along a hinge side, the hinge positionable proximate the first knee compartment for permitting flexion and extension knee movement of the wearer's knee;
a first knee strap having first and second end portions, the first end portion of the first knee strap attachable to the upper leg support, the first knee strap adapted to extend above the knee and wrap around the back of the knee, the second end of the first knee strap attachable to the lower leg support; and
a second knee strap having first and second end portions, the first end portion of the second knee strap attachable to the lower leg support, the second knee strap adapted to extend under the kneecap and wrap around the back of the knee, the second end portion of the second knee strap attachable to the upper leg support, said first and second knee straps for crossing proximate the second knee compartment and providing a corrective unloading force to the first knee compartment of the leg of a person wearing the knee brace.

2. The device of claim 1 wherein the first knee compartment does not contact the knee brace.

3. The device of claim 1 further comprising means for aligning the hinge mechanism with the person's forward progression.

4. The device of claim 1 wherein the first knee compartment is selected from the group consisting of the medial knee compartment and the lateral knee compartment.

5. The device of claim 1 wherein the hinge further comprises means for restricting flexion and extension movement.

6. A knee brace to be worn by a person for unloading pressure to the person's knee having first and second knee compartments comprising:
an upper leg support;
a lower leg support having an upper arm and a lower arm pivotally attached to the upper arm by a pivot joint for permitting selective adjustment in the coronal plane of the lower arm relative the upper arm when the brace is applied to the person's leg;
a hinge connecting the upper and lower leg supports along a hinge side, the hinge positionable proximate the first knee compartment for permitting flexion and extension knee movement of the wearer's knee; and
a knee strap having first and second end portions, the first end portion attached to the upper leg support, the knee strap adapted to extend around the knee and attach to the lower leg support, for providing a varus/valgus correction to the person's knee when normally worn by a person.

7. The device of claim 6 wherein the brace applies a corrective unloading force to the person's first knee compartment.

8. The device of claim 6 wherein the pivot joint further comprises opposing intermeshing teeth.

9. The device of claim 6 wherein the first knee compartment is selected from the group consisting of the medial knee compartment and the lateral knee compartment.

10. The device of claim 7 further comprising a second knee strap having first and second end portions, the first end portion of the second knee strap attachable to the lower leg support, the second knee strap adapted to extend around the knee, and attach to the upper leg support, the first and second knee straps positionable to cross proximate the second knee compartment when the device is normally worn by a person.

11. The device of claim 6 wherein the upper leg support further comprises an upper leg cuff pivotally attached to an upper elongated member to permit rotational adjustment of the upper leg cuff relative to the upper elongated member and the lower arm further comprises a lower leg cuff pivotally attached to a lower elongated member to permit rotational adjustment of the lower leg cuff relative to the lower elongated member.

12. The device of claim 11 wherein the hinge mechanism is aligned with the person's forward progression by adjusting the upper and lower leg cuffs relative to the respective upper and lower elongated members.

13. A knee brace to be worn by a person for encouraging proper alignment of the leg comprising:
an upper leg support having an upper elongated member pivotally attached to an upper leg cuff permitting rotational adjustment of the leg cuff relative to the upper elongated member;
a lower leg support having an elongated member pivotally attached to a lower leg cuff permitting rotational adjustment of the lower leg cuff relative to the lower elongated member;
a hinge mechanism connecting the upper and lower leg supports along a hinge side, the hinge mechanism positionable proximate a first knee compartment for permitting flexion and extension knee movement of the wearer's knee; and
a knee strap having first and second end portions, the first end portion of the knee strap attachable to the upper leg support, the knee strap adapted to extend around the knee, the second end portion of the knee strap attachable to the lower leg support, wherein the brace provides a corrective unloading force to the first knee compartment and the hinge mechanism is aligned with the person's forward progression.

14. The device of claim 13 further comprising a slot extending along a portion of the upper elongated member for adjustably positioning the upper leg support along the length of the upper elongated member and a slot extending along a portion of the lower elongated member for adjustably positioning the lower leg support along the length of the lower elongated member.

15. The device of claim 13 wherein the upper leg cuff and the lower leg cuff further comprise a skin adhesion layer.

16. The device of claim 15 where the skin adhesion layer is a silicone material.

17. A method of applying a corrective unloading force to first and second knee compartments of a person by applying a knee brace to the person's leg, the knee brace having an upper leg support, a lower leg support and a hinge mechanism pivotally connecting the upper and lower leg supports along a hinge side, the method comprising:
positioning the brace so that the hinge mechanism is proximate the first knee compartment;
extending a first knee strap from the upper leg support along the top of the knee and around the back of the knee to the lower leg support; and
extending a second knee strap from the lower leg support along the bottom of the knee and around the back of the knee to the upper leg support, the second knee strap crossing the first knee strap proximate the second knee compartment.

18. A method of applying a corrective unloading force to a knee compartment of a person and a varus/valgus correction to the leg of the person by applying a knee brace to the person's leg, the knee brace having an upper leg support, a lower leg support having an upper arm pivotally attached to a lower arm, and a hinge mechanism connecting the upper leg support and the lower leg support along a hinge side of the brace, the hinge mechanism permitting flexion and extension knee movement, the method comprising:
positioning the brace so that the hinge is proximate the knee compartment;
adjusting the lower arm relative the upper arm to provide a desired angular position between the upper and lower arms; and
extending a first knee strap from the upper leg support around the knee to the lower leg support.

19. A method of applying a corrective unloading force to a knee compartment of a person by applying a knee brace to the leg of the person, the knee brace having an upper elongated member pivotally attached to an upper leg cuff, a lower elongated member pivotally attached to a lower leg cuff, and a hinge mechanism attaching the upper elongated member to the lower elongated member along a hinge side to permit flexion and extension movement of the person's knee, the method comprising:
positioning the brace so that the hinge is proximate the knee compartment;
aligning the hinge mechanism with the person's forward progression by rotationally positioning the upper leg cuff relative the upper elongated member and the lower leg cuff relative the lower elongated member; and
extending a first knee strap from the upper leg support around knee to the lower leg support.

20. A method for encouraging the proper alignment of a leg of a person by applying a knee brace to the person's leg, the knee brace having an upper elongated member pivotally attached to an upper leg cuff, a lower elongated member pivotally attached to a lower leg cuff, and a hinge mechanism attaching the upper elongated member to the lower elongated member along a hinge side to permit flexion and extension movement of the person's knee, the lower elongated member having an upper arm pivotally attached to a lower arm, the method comprising:
positioning the brace so that the hinge is proximate a first knee compartment of the person's knee;
adjusting the lower arm relative the upper arm to provide a desired angular position between the upper and lower arms;
aligning the hinge mechanism with the person's forward progression by rotationally positioning the upper leg cuff relative the upper elongated member and the lower leg cuff relative the lower elongated member; and
extending a first knee strap from the upper leg support around the knee to the lower leg support.
